(19) United States
(12) Patent Application Publication
(10) Pub. No.: US 2004/0076614 A1
(43) Pub. Date: Apr. 22, 2004

(54) Symbiotic regenerative agent

(76) Inventor: Jorg P Schur, Wegberg-Dalheim (DE)

Correspondence Address: JENKINS & WILSON, PA, 3100 TOWER BLVD, SUITE 1400, DURHAM, NC, 27707, US

(21) Appl. No.: 10/467,040

(22) PCT Filed: Feb. 1, 2002
(86) PCT No.: PCT/EP02/01056

(30) Foreign Application Priority Data
Feb. 2, 2001 (EP) 01102384.3

(51) Int. Cl. A01N 63/00; A61K 35/76

(52) U.S. Cl. 424/93.4; 424/780

(57) ABSTRACT

The present invention relates to a regenerative medicament for the natural infection-immunological therapy with induction, regulation and effector mechanisms for the early natural cellular immune defense in humans and animals, comprising a special composition with a microorganism component and a modulator component, to food, food supplements, animal feeds and animal feed supplements containing such composition, and to the use of such composition for the preparation of regenerative agents for the treatment of humans and animals.

The present invention relates to a regenerative medicament for the natural infection-immunological therapy with induction, regulation and effector mechanisms for the early natural cellular immune defense in humans and animals, comprising a special composition with a microorganism component and a modulator component, to food, food supplements, animal feeds and animal feed supplements containing such composition, and to the use of such composition for the preparation of regenerative agents for the treatment of humans and animals.

### BACKGROUND OF THE INVENTION

The large intestine is highly colonized with microorganisms (10¹⁰ to 10¹² per g of contents); it is the most highly colonized portion of the human or animal digestive tract and of the whole body. In the small intestine, there are fewer microorganisms (about 10⁴ to 10⁸ organisms/ml). The pH value of 1-2 in the stomach virtually does not allow any life of bacteria, so that only 10¹ to 10³ organisms/ml live there (Organismen im Darmtrakt, Tannock, 1995).

Thus, the acidic environment in the stomach is an important barrier against pathogens and foreign organisms. These also include the so-called “probiotics”, i.e., single types of microorganisms and/or mixtures, mostly from the group of “apathogenic” lactobacilli (acidophilus, bifidus and the like), which have recently been offered and used and food supplements, sometimes also in combinations with vitamins and/or minerals/trace elements or complexes thereof. It has been established that so-called “probiotic strains” (apathogenic microorganisms, e.g., the group of lactobacilli) have great difficulty to settle in the intestine, i.e., there is a colonizing resistance or barrier effect (Tannock, 1995).

Thus, it is doubtful whether a single strain or mixtures from a single group (apathogenic lactobacilli) exert a favorable influence or an activation of immune defense (Tannock, 1998).

Since the natural intestinal flora plays an important role in the defense system of humans and animals against undesirable pathogens and other microorganisms, this is particularly evident when it is disturbed by malnutrition and/or administration of medicaments.

Malnutrition, application of anti-infective agents, immunosuppressants, antidepressants, contraceptives or anti-allergic agents causes, on the one hand, promotion of resistances in microorganisms and, on the other hand, generation of associated diseases whose symptoms are often not associated causally therewith, because they mimic other clinical pictures. Thus, for example, “fungal infections”, e.g., of the genera Candida and Aspergillus, have significantly increased in recent years, and the above mentioned clinical pictures are with high probability to be considered causally related with Candida and/or Aspergillus contaminations in the mucosa and gastrointestinal tract, all the more so since yeasts and fungi of these genera are also capable of forming mutagenic and cancerogenic toxins, which in turn have effects which in part not only reduce the quality of food, but are even life-threatening. As early as in 1980, H. J. Preusser (Medical Mycology Zbl. Bact. Suppl. 8) describes the importance of the “pathogenic potential in the genus Candida”, and R. Hurley/De Louvois report on the “ecological aspects of yeast-like fungi of medical importance”. In Wien. Klin. Wochenschr. 91, 826-830 (1979), O. Male/Boltz-Nitulescu already described animal trial studies on the persorption of Candida albicans and the possibility of intestinal triggering of an immune response. Th. Büchner reports in “Pilzinfektionen in der Onkologie”, Schattauer, 1996, on predisposing factors for mycoses: hormonal diseases, therapy: corticosteroids; gastroenterological diseases: therapy immunosuppressants; hematological diseases, therapy: cytostatic agents; immunodeficiencies (including AIDS), therapy: irradiation; malignant tumors, therapy: antibiotics; infectious diseases, chronic diseases, burns, macrosurgery.

The susceptibility for diseases and/or infections is increased in practice by the said malnutrition and the application of anti-infective agents. Thus, the intestinal flora has one of the most important protective functions in the body:

ousting of health-hazarding bacteria by competition in situ (colonization resistance/barrier effect);

production of organic acids (lactic acid, acetic acid, butyric acid, propionic acid) and bacteriocins, which have a microbicidal effect;

stimulation of the immune system by increasing the activity of killer cells, lymphocytes, macrophages (cellular immune response) and increased production of cytokines (secretion) and antibodies (humoral immune response).

Thus, for example, it has been established in studies that cancer activity (e.g., in malignant tumors) decreased when more immune reactions occurred with increased T-cell stimulation and cytokine secretion (K. F. Kölmel, Melanoma Research 2, pp. 207-211 (1992)). Since “normal” medicaments, such as immunosuppressants (e.g., corticosteroids), weaken the formation of T lymphocytes, reduce the number of eosinophils (special white blood cells) and suppress T-suppression cells, it is clear that, in view of today's medication (e.g., allergies) with these and other preparations which more or less strongly influence the immune system and/or enable or even provoke a “foreign population” (e.g., selectionism) in the gastrointestinal tract, e.g., from mycoses, and their highly dangerous toxin productions (e.g. Aspergillus parasiticus—aflatoxin—cancerogenic toxin) (Ziemer and Gibson, 1998), we have meanwhile become in need of a new generation of regenerative medicaments which exert their activity and accomplish their tasks in a natural and causal way.

60% of the total antibodies of the human and animal organisms is prepared in the gastrointestinal tract (Scientific Concepts of Functional Food in Europe, 1999). The pathogens which have overcome the defense mechanisms in the nasal, oral and pharyngeal cavities and the esophagus, the stomach (pH 1-2), bile acid and enzymes of the pancreas cannot settle anywhere in the healthy body since the intestine is so highly colonized with the body's own microorganism lawn that there is little space for “docking”. Macrophages and natural killer (NK) cells are essential elements of the first line of defense, i.e., the natural non-antigen-specific cellular infection defense.

In the healthy organism, these elements have a great influence on the intensity of the subsequent adaptive antigen-specific defense phase. The natural immune system is always capable of a reactivating defense performance, in contrast to adaptive—acquired or genetically caused—immunodeficiency. Even in the absence of functional T and B cells, the immune system can produce macrophages (cellular) and activating interferon (γ) and thus inhibit bacterial replication “post infectionem” with, for example, facultative intracellular bacteria, such as Listeria monocytogenes. The source thereof is the natural killer cells, activated by pathogen-stimulated macrophages. This mechanism of pathogen-induced activation of the natural cellular defense could be confirmed with a wide variety of microorganisms. The interaction of macrophages and natural killer cells is probably mediated, not by cytokine secretion, but also by ligands in the membrane.

As to the rest, macrophages, which occur in different regions of the organism, are the “eating cells” (antigen-presenting or antigen-producing) of the defense system, but some pathogens may also survive in these macrophages and multiply in the cellular system. The killing of pathogens ingested in macrophages is effected by the formation of reactive oxygen free radicals, e.g., by cytokines or their genes (humoral=(re)activation of macrophages). Activated macrophages may also be tumoricidal.

In order to train the immune system, a continuous metabolic transport from the intestinal mucosal tissue, which belongs to the lymphatic defense system, into the body (body cells) must occur to activate the body's own immune system. Also, beneficial bacteria nourish the mucosa, for example, with vitamins etc. for regulating the intestinal activity. Thus, a regenerative immune regulation with symbiotic microorganisms and corresponding necessary substances appears to be appropriate and effective, all the more so since the nose, mouth, throat, small intestine, large intestine, genital and mucosal regions in the human and animal bodies are colonized with microorganisms necessary to life (about 100 trillions). From 400 to 500 different species are living here; in the large intestine, these are predominantly Bacteroides, Eubacterium, Bifidobacterium, Clostridium, Enterococcus, Fusobacterium, Peptostreptococcus, Ruminococcus, Lactobacillus and Escherichia coli.

In the prior art, compositions in which a viable culture of microorganisms is an essential component have already been known (as medicaments or food supplements). Thus, the food supplement “Merck Bion 3” contains 3 kinds of lactobacilli (acidophilus, bifidus, bifidolongum), 13 vitamins and 14 minerals and trace elements (see DE-A-19830528 and EP-A-0 931 543). In The Lancet, Vol. 354, 635-639 (1999), B. J. Rembach et al. report that colitis ulcerosa (“IBO, inflammatory bowel disease”) was successfully treated with E. coli microorganisms. Further, K. F. Kölmel in Melanoma Res., Vol. 9, p. 511-519 (1999), reports that the administration of a lysate of streptococci and Serratia marescens results in an increased immunity in the defense/reduction of malignant (skin) tumors with rise of fever, TNF-α level in the serum, concentration of thrombin/antithrombin III complex/fibrin (case studies).

EP-A-0 904 784 (Unilever Research) discloses that a fungicidal protection and lower fungal infestation (perishing) of food can be achieved by treating the food with various lactobacilli with or without one or more yeasts (e.g., Saccharomyces cerevisiae, GRAS strains, pediococci, propioni bacteria or leuconosto) singly or in combination. Food or quasi-medications for the improvement of gastro-intestinal problems are also mentioned.

WO 99/49875 reports on the controlling of intestinal problems or clinical pictures by the administration of antibiotics. Thus, lactobacilli, Saccharomyces (yeasts) and enterococci are administered in common and in combination with the antibiotics in order to reduce or improve their intestinal side effects.

The above mentioned application DE 198 30 528 relates to multilayer tablets which comprise one or more probiotic microorganisms in one layer and nutrition-relevant additives in a further layer. Due to the separation of the components in this multilayer tablet, the viability of the microorganisms is to be ensured, which is an ineffective measure, however, according to our knowledge.

Although it is mentioned for some of the above described compositions that the administration of microorganisms is accompanied by some pharmacological/gastro-intestinal effect, this effect is not so strong that an actual regeneration of an impaired human or animal organism is possible. Examples thereof are administrations of partially pathogenic microorganisms, such as E. coli, streptococci and Serratia pathogens, which, much like vaccinations, exclusively cause an antibody/antigen reaction with possible accompanying phenomena (e.g., fever, indisposition and the like) and necessitate clinical attendance. A “standard” medication is not possible with this.

Now, it is the object of the present application to provide a medicament or food supplement which has a regenerative effect on impaired cultures of microorganisms and their functions (or impaired metabolism) in humans and animals. The medicament or food supplement should also have sufficient stability, i.e., a sufficient number of living microorganisms should be present in the final dosage form. Surprisingly, within the scope of the developmental work and bacteriological tests relating to the parallel application EP 00 124 497, it was observed that certain substances, such as GRAS flavoring agents, not only have protagonistic decontaminative microbicidal properties, but are also capable of doing the very opposite, namely display antagonistic regenerative properties, i.e., stabilize or preserve or even promote the growth of microorganisms. Based on these findings, it has now been found that impaired cultures of microorganisms and their functions (or impaired metabolism) can be regenerated by the administration of a combination of a special microorganism component and a special modulator component.

The effect of the medicament according to the invention (also referred to as “symbiotic regenerative agent in the following) is based on the fact that synergisms of groups of substances in connection with microorganisms (of all kinds, if possible) exist which have a growth-promoting stimulating and colonizing property, so that these microorganisms are “docking” where they may be employed and/or needed, and do not die in the course of their activity, but increasedly develop their properties there, i.e., can permeate or penetrate. On the one hand, it is possible to cause beneficial microorganisms, such as probiotics (lactobacilli) to colonize there, so that they are capable to restore a healthy medium. On the other hand, facultatively pathogenic and/or pathogenic microorganisms can thereby be effectively “docked” in small amounts (with or without beneficial microorganisms) to the site of therapeutic necessity to display their activity. In this case, this may also be a weakly manifested antibody/antigen activity.

It has been found that both flavoring agents or their synergistic mixtures and enzymes or their synergistic mixtures or plant substances or their synergistic mixtures enhance such colonizations, stimulations or docking functions by symbiotic synergisms, i.e., develop regenerative properties in connection with the microorganisms.

### SUMMARY OF THE INVENTION

Accordingly, the present application relates to:

(1) a regenerative medicament comprising:

(a) a microorganism component containing:

(a1) at least one type of apathogenic microorganisms; and/or

(a2) at least one type of pathogenic or facultatively pathogenic microorganisms;

(b) a modulator component containing

(b1) at least one enzyme;

(b2) at least one GRAS (generally recognized as safe) flavoring agent or a derivative thereof; and/or

(b3) at least one animal or plant extract or derivative thereof which comprises a compound with alcoholic, acid, ester, aldehyde, acetal, phenolic functional groups and/or with double bonds;

(2) a preferred embodiment of the medicament (1), wherein

(i) the microorganism component exclusively consists of apathogenic microorganisms (a1); and/or

(ii) the modulator component contains at least one GRAS flavoring agent or derivative thereof;

(3) a preferred embodiment of the medicament (1) or (2), wherein the medicament can be applied by inhalation, orally, intravenously, intramuscularly, rectally, as a contact preparation, internally/externally (also mucosa), intraperitoneally, subcutaneously, on/in internal organs (e.g., by endoscopy) in the form of tablets, liquid, gas, powder, injection, infusion, suppository, spray, ointments or plasters;

(4) the use of the composition as defined under (1) or (2) consisting of a microorganism component (a) and a modulator component (b) for the preparation of a decontaminative and/or regenerative agent, especially an immune regulator, immunoregenerative agent, anti-infective agent, antimycotic agent, antitoxinocidic agent, antiresistance agent or cytostatic agent, for the treatment of humans and animals;

(5) a method for the treatment of non-specific genesis, allergies, cancer diseases (also in combination with known therapies, such as chemotherapies and the like), symptoms which are not causally associated with clinical pictures susceptible to regenerative therapy (such as depressions, rheumatism, arthritis, vegetative symptoms, overweight, bronchial diseases etc.), of immune diseases and infectious diseases in humans and animals, comprising the administration of a composition consisting of a microorganism component (a) and a modulator component (b) as defined above under (1) or (2);

(6) a food, food supplement, animal feed or animal feed supplement comprising a composition consisting of a microorganism component (a) and a modulator component (b) as defined above under (1) or (2); and

(7) a method for the stabilization of cultures of microorganisms, comprising the treatment, storage and/or culturing of said culture of microorganisms with or in the presence of at least one GRAS flavoring agent or derivative thereof as defined under (1) or (2).

The medicaments defined above under (1) to (3) can be employed as regenerative agents for maintaining or supporting or promoting the growth of microorganisms necessary to life (e.g., in the gastro-intestinal tract, small intestine, oral mucosa, genital mucosa), both

(A) as an agent having a general regenerative effect; and/or

(B) in multi-step applications (e.g., as step 2 after decontaminative application (e.g., as described in EP 00 124 497) in step 1); and/or

(C) in combination with known medicaments, food, fodders or supplements.

The use of these agents (A), (B) and (C) as a regenerative agent is effected in/on the human or animal body and may be applied individually, in combinations or in a multi-step method. It is solid, liquid or gaseous. When applied orally, it may be supplemented with inert additives/carrier agents/fillers/supplements to display its selective activity and the like in the corresponding passages of the stomach, small intestine and large intestine or ileum, also with timers combined therewith, i.e., functionally inert substances for the well-aimed application fixed in time. Other applications include: i.m., i.v., inhalational, oral, intravenous, intramuscular, rectal, contact preparation, internal/external (also mucosa), intraperitoneal, subcutaneous, on/in internal organs (e.g., by endoscopy) in the form of tablets, liquid, gas, powder, injection, infusion, suppository, spray, ointments, plasters.

The medicament (1) or (3) or the agent (5) regeneratively supply the body with microorganisms (e.g., lactobacilli, Bac. subtilis, positive coliform bacteria and the like), which are then preserved, stabilized or enhanced in their body cell adaptation and in their function and proliferated, so that a regenerative effect necessary to the body is possible. Given about 40 trillions of body cells, but 100 trillions of microorganisms (about 400 to 500 species), this is an altogether plausible and necessary process, all the more so since the equilibrium of the necessary species of microorganisms present in the body and their quantity and quality (resistances or selectionism, i.e., other germs overgrow or increase) in many humans and animals is already disturbed due to the way of living and the medication of recent times, which is the cause of many clinical pictures as described above.

### DETAILED DESCRIPTION OF THE INVENTION

“Microorganisms” within the meaning of the present invention comprises fresh (vital) cells with or without substrates, lysates, killed cells, frozen, deep-frozen, cooled, preserved etc. microorganisms, each of them with or without carrier or separating agents.

The apathogenic microorganism (a1) is preferably an apathogenic lactobacillus, preferably selected from Lactobacillus acidophilus, Lactobacillus bifidum, Lactobacillus bulgaris, Lactobacillus casei, Lactobacillus bifidolongum and Lactobacillus fermentum, and/or a microorganism selected from fungi, dermatophytes and yeasts which do not have pathogenic or toxinogenic properties or which occur in the gastro-intestinal tract, including Sarcina, Staphylococcus epidermis, Gaffkya, Streptococcus K-P, Corynebacterium xerosis, Corynebacterium hoffmanni, Bacillus subtilis and Saccharomyces cerevisiae.

The pathogenic or facultatively pathogenic microorganisms (a2) are preferably Serratia marcescens, Alcaligenes faecalis, Citrobacter, Hafnia alvei, Afflya tetragena, Neisseria catarrhalis, Neisseria sicca, Neisseria flavescens, subflava, flava, perflava, Veillonella parvula, Borrelia buccalis, Rhodotorula rubra, Cladosporium, Geotrichum mucor, Mucor and Torulopsis.

In addition to the organisms which can be clearly classified as apathogenic, pathogenic or facultatively pathogenic, those microorganisms which currently cannot be assigned to one of these classifications or which are pathogenic or apathogenic depending on the organism can also be employed. These are, for example, certain Lactobacillaceae (a3), certain Eubacteriales (a4) and certain Enterobacteriaceae as well as the large group of yeasts, fungi, dermatophytes and viruses.

The Lactobacillaceae (a3) include Streptococcus A-T, Streptococcus viridans I-IV, Streptococcus salivarius, Peptostreptococcus and Diplococcus pneumonia.

The Eubacteriales (a4) include Alkaligenes, Achromobacter, Flavobacterium, Escherichia, Shigella, Salmonella, Arizona, Klebsiella, Enterobacter, Serratia proteus, Proviridencia, Edwardsiella, Pasteurella, Yersinia, Francisella, Bordetella, Brucella, Haemophilus, Moraxella, Actinibacillus, Fusobacterium bacteroides, Fusobacterium spaerophorus, Streptobacillus, Staphylococcus, Micrococcus, Sarcina, Peptococcus, Neisseria, Veillonella, Streptococcus, Peptostreptococcus, Diplococcus, Corynebacterium, Listeria, Erysipelothrix, Bacillus and Clostridium.

The Enterobacteriaceae (a5) include Enterobacteriaceae, Brucellaceae, Micrococcaceae, Corynebacteriaceae, Bacillaceae, Actinomyceaceae, Treponemataceae, Mycoplasmataceae, Bartonellaceae, Achromobacteriaceae and Pseudomonadaceae.

As the enzyme (b1) according to the present invention, enzymes of animal or synthetic origin can be used. It is particularly preferred according to the present invention for the enzyme to be selected from chymotrypsin, bromelain, papain, pepsin, trypsin, an enzyme from the groups of oxidoreductases, dehydrogenases, oxygenases, transferases, hydrolases, esterases, glycosidases, proteases, lyases, isomerases, ligases, synthetases, DNA ligases, amylases, lipases, phosphatases, leucoproteases, cathepsins, oxidases, such as catalases and peroxidases, carboxylases, more preferably a hydrolase or proteases, such as bromelain, trypsin, ananain, pepsin, chymosin and ficain.

The GRAS (generally recognized as safe) flavoring agents (b2) are recognized by the FDA authority as commercially safe for use in foods (GRAS=generally recognized as safe in food). The mentioned GRAS flavoring agents are the compounds mentioned in the FEMA/FDA GRAS Flavour Substances Lists GRAS 3-15 Nos. 2001-3905 (as of 2000). This list contains natural and synthetic flavoring agents approved by the American public health authority, FDA, for use in food-stuffs: FDA Regulation 21 CFR 172.515 for synthetic flavoring agents (Synthetic Flavoring Substances and Adjuvants) and FDA Regulation 21 CFR 182.20 for natural flavoring agents (Natural Flavoring Substances and Adjuvants).

The function of component (b2) is based on the following new principle of action: The composition permits penetration of the components into the microorganism and thereby prevents its proliferation, but does not destroy it. The regenerative activity permits penetration into the microorganism and/or the body cell to thereby stabilize and/or to activate the metabolism of and/or to proliferate (colonize) and/or permeate “benign” microorganisms.

The GRAS flavoring agents (b2) of the present invention are preferably selected from (i) GRAS flavor alcohols or their derivatives, (ii) GRAS polyphenols, (iii) GRAS acids or their derivatives, (iv) GRAS phenols or their derivatives, (v) GRAS esters, (vi) GRAS terpenes, (vii) GRAS acetals, (viii) GRAS aldehydes and (ix) GRAS essential oils. Preferably, at least two GRAS flavoring agents are employed.

In detail, the following GRAS flavor alcohols (i) may be employed, for example: benzyl alcohol, acetoin (acetylmethylcarbinol), ethyl alcohol (ethanol), propyl alcohol (1-propanol), iso-propyl alcohol (2-propanol, isopropanol), propylene glycol, glycerol, n-butyl alcohol (n-propyl carbinol), iso-butyl alcohol (2-methyl-1-propanol), hexyl alcohol (hexanol), L-menthol, octyl alcohol (n-octanol), cinnamyl alcohol (3-phenyl-2-propene-1-ol), α-methylbenzyl alcohol (1-phenyl-ethanol), heptyl alcohol (heptanol), n-amyl alcohol (1-pentanol), iso-amyl alcohol (3-methyl-1-butanol), anisalcohol (4-methoxybenzyl alcohol, p-anisalcohol), citronellol, n-decyl alcohol (n-decanol), geraniol, β-γ-hexanol (3-hexenol), lauryl alcohol (dodecanol), linalool, nerolidol, nonadienol (2,6-nonadiene-1-ol), nonyl alcohol (nonanol-1), rhodinol, terpineol, borneol, clineol (eucalyptol), anisole, cuminyl alcohol (cuminol), 10-undecen-1-ol, 1-hexadecanol. As said derivatives, both natural and synthetic (naturally occurring or not) derivatives can be employed. Suitable derivatives include, for example, the esters, ethers and carbonates of the above mentioned GRAS flavor alcohols. Particularly preferred GRAS flavor alcohols are benzyl alcohol, 1-propanol, glycerol, propylene glycol, n-butyl alcohol, citronellol, hexanol, linalool, acetoin and their derivatives.

As polyphenols (ii), the following polyphenols may be employed, in particular: catechol, resorcinol, hydroquinone, phloroglucinol, pyrogallol, cyclohexane, usnic acid, acylpolyphenols, lignins, anthocyans, flavones, catechols, gallic acid derivatives (e.g., tannins, gallotannin, tannic acids, gallotannic acids), carnosol, carnosolic acid (including their derivatives, such as (2,5-dihydroxyphenyl)carboxylic and (2,5-dihydroxyphenyl)alkylenecarboxylic substitutions, salts, esters, amides), caffeic acid and its esters and amides, flavonoids (e.g., flavone, flavonol, isoflavone, gossypetin, myricetin, robinetin, apigenin, morin, taxifolin, eriodictyol, naringin, rutin, hesperidin, troxerutin, chrysin, tangeritin, luteolin, catechols, quercetin, fisetin, kaempferol, galangin, rotenoids, aurones, flavonols, diols), extracts, e.g., from Camellia, Primula. Further, their possible derivatives, e.g., salts, acids, esters, oxides and ethers, may also be used. A particularly preferred polyphenol is tannin (a GRAS compound).

As GRAS acids (iii), the following acids may be used, for example: acetic acid, aconitic acid, adipic acid, formic acid, malic acid (1-hydroxysuccinic acid), capronic acid, hydrocinnamic acid (3-phenyl-1-propionic acid), pelargonic acid (nonanoic acid), lactic acid (2-hydroxypropionic acid), phenoxyacetic acid (glycolic acid phenyl ether), phenylacetic acid α-toluenic acid), valeric acid (pentanoic acid), iso-valeric acid (3-methylbutyric acid), cinnamic acid (3-phenylpropenoic acid), citric acid, mandelic acid (hydroxyphenylacetic acid), tartaric acid (2,3-dihydroxybutanedioic acid; 2,3-dihydroxysuccinic acid), fumaric acid, tannic acid and their derivatives.

Suitable derivatives according to the present invention are esters (e.g., C₁₋₆-alkyl esters and benzyl esters), amides (including N-substituted amides) and salts (alkali, alkaline earth and ammonium salts) of the above mentioned acids. According to the present invention, the term “derivatives” also encompasses modifications of the side-chain hydroxy functions (e.g., acyl and alkyl derivatives) and modifications of the double bonds (e.g., the perhydrogenated and hydroxylated derivatives of the mentioned acids).

As GRAS phenols (iv), the following phenol compounds may be employed: thymol, methyleugenol, acetyleugenol, safrol, eugenol, isoeugenol, anethole, phenol, methylchavicol (estragol; 3-(4-methoxyphenyl)-1-propene), carvacrol, α-bisabolol, formesol, anisole (methoxybenzene), propenylguaethol (5-propenyl-2-ethoxyphenol) and their derivatives. Derivatives according to the present invention are compounds in which the phenolic hydroxy group has been esterifled or etherified.

As GRAS esters (v), allicin and the following acetates may be used, for example: iso-amyl acetate (3-methyl-1-butyl acetate), benzyl acetate, benzylphenyl acetate, n-butyl acetate, cinnamyl acetate (3-phenylpropenyl acetate), citronellyl acetate, ethyl acetate (acetic ester), eugenol acetate (acetyleugenol), geranyl acetate, hexyl acetate (hexanyl ethanoate), hydrocinnamyl acetate (3-phenyl-propyl acetate), linalyl acetate, octyl acetate, phenylethyl acetate, terpinyl acetate, triacetin (glyceryl triacetate), potassium acetate, sodium acetate and calcium acetate. Further suitable esters are the ester derivatives of the above defined acids (iii).

As terpenes (vi), there may be used, in particular, camphor, limonene and β-caryophyllene.

The acetals (vii) which can be used include, in particular, acetal, acetaldehyde dibutyl acetal, acetaldehyde dipropyl acetal, acetaldehyde phenethyl propyl acetal, cinnamic aldehyde ethylene glycol acetal, decanal dimethyl acetal, heptanal dimethyl acetal, heptanal glyceryl acetal and benzaldehyde propylene glycol acetal.

As aldehydes (viii), there may be used, in particular, acetaldehyde, anisaldehyde, benzaldehyde, iso-butyl aldehyde (methyl-1-propanal), citral, citronellal, n-caprylic aldehyde (n-decanal), ethylvanillin, furfurol, heliotropin (piperonal), heptyl aldehyde (heptanal), hexyl aldehyde (hexanal), 2-hexenal (β-propyl-acrolein), hydrocinnamic aldehyde (3-phenyl-1-propanal), lauryl aldehyde (dodecanal), nonyl aldehyde (n-nonanal), octyl aldehyde (n-octanal), phenylacetaldehyde (1-oxo-2-phenylethane), propionaldehyde (propanal), vanillin, cinnamic aldehyde (3-phenylpropenal), perillaldehyde and cuminaldehyde.

As GRAS essential oils (ix), the following essential oils and/or alcoholic or glycolic extracts or extracts obtained by CO₂ high-pressure processes from the mentioned plants may be employed, in particular:

(ix1) oils or extracts having a high content of alcohols: melissa, coriander, cardamon, eucalyptus;

(ix2) oils or extracts having a high content of aldehydes: Eucalyptus citriodora, cinnamon, lemon, lemon grass, melissa, citronella, lime, orange;

(ix3) oils or extracts having a high content of phenols: origanum, thyme, rosemary, orange, clove, fennel, camphor, mandarin, anise, cascarilla, estragon and pimento;

(ix4) oils or extracts having a high content of acetates: lavender;

(ix5) oils or extracts having a high content of esters: mustard, onion, garlic;

(ix6) oils or extracts having a high content of terpenes: pepper, bitter orange, caraway, dill, lemon, peppermint, nutmeg.

In a preferred embodiment of the present invention, component (b2) contains one or more GRAS flavor alcohols or their derivatives. According to the invention, it is preferred to use one, two or three GRAS flavor alcohols.

In a further preferred embodiment, (b2) contains:

benzyl alcohol as a necessary component; and optionally

one or more further GRAS flavor alcohols (i) or their derivatives; and

one or more polyphenol compounds (ii); and/or

one or more GRAS acids (iii) or their derivatives.

In this embodiment, the above defined GRAS flavoring agents (iv) to (ix) may further be contained in (b2). Particularly preferred among the further GRAS flavoring agents are the phenols (iv), aldehydes (viii) and essential oils (ix).

Particularly preferred according to the present invention are those components (b2) which exclusively consist of GRAS flavoring agents, i.e., does not contain any “derivatives” of the GRAS flavoring agents. As an example of such a composition, there may be mentioned a mixture of benzyl alcohol, one or two of the above mentioned GRAS flavor alcohols (i) and tannin. Another example is a mixture of two alcohols (i), a polyphenol (especially tannin) and an essential oil (ix), especially the phenolic essential oil (ix3).

In another preferred embodiment, (b2) contains at least two GRAS essential oils (ix). These are preferably the above mentioned essential oils and/or alcoholic or glycolic extracts or extracts obtained by CO₂ high-pressure processes, (ix1) to (ix6). In addition, they may contain further GRAS flavoring agents, such as alcohols (i), polyphenol compounds (ii), acids (iii), phenols (iv), esters (v), terpenes (vi), acetals (vii), aldehydes (viii) or their derivatives. Particularly preferred compositions of this embodiment contain at least three GRAS essential oils (ix) and/or contain the further GRAS flavoring agents anisol and quercitin in addition to the essential oils (ix).

In another preferred embodiment, (b2) contains at least one lipophilic and at least one hydrophilic GRAS flavoring agent (however, it is to be noted that hydrophilic-hydrophilic and lipophilic-lipophilic GRAS flavoring agent combinations are also suitable for the medicaments according to the invention). The hydrophilic GRAS flavoring agent may be a hydrophilic alcoholic GRAS flavoring agent (iₕ) and/or a hydrophilic non-alcoholic GRAS flavoring agent. Preferred are those compositions which further contain benzyl alcohol and/or a polyphenol compound (ii) in addition to the mentioned hydrophilic compounds.

The hydrophilic alcoholic GRAS flavoring agents (iₕ) are monovalent or polyvalent alcohols having from 2 to 10 carbon atoms, preferably from 2 to 7 carbon atoms. Particularly preferred compounds are 1-propanol, glycerol, propylene glycol and acetoin. Hydrophilic non-alcoholic GRAS flavoring agents are selected from organic acids (iiiₕ) having from 1 to 15 carbon atoms and physiologically acceptable salts thereof, hydrophilic acetates (vₕ) and hydrophilic aldehydes (viiiₕ). Preferred organic acids (iiiₕ) are those having from 2 to 10 carbon atoms, especially acetic acid, aconitic acid, formic acid, malic acid, lactic acid, phenylacetic acid, citric acid, mandelic acid, tartaric acid, fumaric acid, tannic acid, hydrocinnamic acid and their physiologically acceptable salts. The hydrophilic acetate (vₕ) is preferably selected from allicin, triacetin, potassium acetate, sodium acetate and calcium acetate, and the hydrophilic aldehyde (viiiₕ) is preferably selected from furfurol, propionaldehyde and vanillin.

In this embodiment, the lipophilic GRAS flavoring agents are preferably selected from (iₗ) lipophilic GRAS flavor alcohols or their derivatives, (ii) polyphenol compounds, (iiiₗ) lipophilic GRAS flavor acids or their derivatives, (iv) phenols or their derivatives, (vₗ) lipophilic esters, (vi) terpenes, (vii) acetals, (viiiₗ) lipophilic aldehydes and (ix) essential oils. In this embodiment, (c5) preferably contains two of the mentioned lipophilic GRAS flavoring agents.

Suitable lipophilic GRAS flavor alcohols (iₗ) among the above defined alcohols (i) are, in particular:

aromatic GRAS flavor alcohols, comprising benzyl alcohol, 2-phenylethanol, 1-phenylethanol, cinnamyl alcohol, hydrocinnamyl alcohol, 1-phenyl-1-propanol and anisalcohol; and aliphatic GRAS flavor alcohols, comprising n-butyl alcohol, iso-butyl alcohol, hexyl alcohol, L-menthol, octyl alcohol, heptyl alcohol, n-amyl alcohol, iso-amyl alcohol, anisalcohol, citronellol, n-decyl alcohol, geraniol, β-γ-hexanol, lauryl alcohol, linalool, nerolidol, nonadienol, nonyl alcohol, rhodinol, terpineol, borneol, clineol, anisole, cuminyl alcohol, 10-undecene-1-ol and 1-hexadecanol and their derivatives. The aromatic GRAS flavor alcohols and especially benzyl alcohol are preferred.

The lipophilic polyphenol compound (ii), phenols or their derivatives (iv), terpenes (vi), acetals (vii) and essential oils (ix) are preferably the above defined compounds (ii), (iv), (vi), (vii) and (ix). The lipophilic GRAS flavor alcohols or their derivatives (iiiₗ), lipophilic esters (vₗ) and lipophilic aldehydes comprise all the specifically mentioned acids, esters and aldehydes except for the compounds (iiiₕ), (vₕ) and (viiiₕ) specifically mentioned above.

In a particularly preferred embodiment, (b2) contains either

two lipophilic GRAS flavor alcohols (iₗ), but no benzyl alcohol and no polyphenol compounds (ii); or

benzyl alcohol and/or a polyphenol compound (ii), but no further GRAS flavor alcohols.

It is particularly preferred for (b2) to contain exclusively non-alcoholic hydrophilic GRAS flavoring agents, especially exclusively a hydrophilic GRAS flavor acid (iiiₕ).

In a further particularly preferred embodiment, (b2) contains

one or more GRAS flavor alcohols (iₗ) or their derivatives; and

one or more flavoring agents selected from polyphenol compounds (ii) and lipophilic GRAS flavor acids or their derivatives (iiiₗ).

In addition, (b2) may contain further GRAS flavoring agents selected from (iv) phenols or their derivatives, (vₗ) lipophilic esters, (vi) terpenes, (vii) acetals, (viiiₗ) lipophilic aldehydes and (ix) essential oils.

Further, in this particularly preferred embodiment, it is preferred for (b2) to contain benzyl alcohol as a necessary component and optionally one or more further lipophilic GRAS flavor alcohols or their derivatives (iₗ). In this case, further lipophilic GRAS flavoring agents (iv) to (ix) as defined above may be contained. Said further lipophilic GRAS flavoring agents are more preferably phenols (iv) and/or essential oils (ix).

In a further particularly preferred embodiment, (b2) contains two lipophilic GRAS flavor alcohols and at least one polyphenol compound (ii), which is preferably tannin.

Particularly preferred according to the present invention are those compositions (b2) which exclusively consist of GRAS flavoring agents, i.e., do not contain any “derivatives” of the GRAS flavoring agents. As an example of such a composition, there may be mentioned a mixture of benzyl alcohol, one or two of the above mentioned GRAS flavor alcohols (aₗ) and tannin. Such a mixture preferably contains from 0.1 to 98% by weight of benzyl alcohol and from 0.01 to 10% by weight, preferably from 1 to 10% by weight, of tannin. Another example of a preferred composition is a mixture of two alcohols, a polyphenol (especially tannin) and an essential oil (especially a phenolic essential oil, component (i3)).

In another preferred embodiment of the present invention, (b2) contains at least one GRAS flavoring agent having a double bond (Δ) or a derivative thereof, preferably at least two such compounds. Examples of compound (Δ) include unsaturated GRAS alcohols (i_{Δ}), such as cinnamyl alcohol, citronellol, 3-hexenol, nonadienol and 10-undecen-1-ol, unsaturated GRAS acids (iii_{Δ}), such as cinnamic acid and fumaric acid, unsaturated GRAS esters (iv_{Δ}), such as cinnamic acid esters (e.g., ethyl cinnamate and propyl cinnamate), cinnamyl acetate and citronellyl acetate, unsaturated GRAS acetals (vii_{Δ}), such as cinnamic aldehyde ethylene glycol acetal, and unsaturated GRAS aldehydes (viii_{Δ}), such as cinnamic aldehyde and citronellal. As further GRAS flavoring agents, these compositions preferably contain essential oils (ix) and/or the above defined hydrophilic GRAS flavoring agents.

The following are particularly preferred compositions (b2). The stated amounts, which are in percent by weight unless otherwise specified, are only particularly preferred embodiments of the respective compositions.

1. Compositions comprising at least two GRAS flavoring agents or their derivatives:

45% anisalcohol (i), 35% borneol (i), 20% rhodinol (i);

82% valeric acid (iii), 18% camphor (ix3);

10% malic acid (iii), 90% acetoin (i);

12% α-bisabolol (iv), 18% geranyl acetate (i), 20% rhodinol (i).

2. Compositions comprising one or more GRAS flavor alcohols (a) and one or more GRAS flavors:

65% propylene glycol (i), 10% caffeic acid (ii), 10% tannin (ii), 5% resveratrol (ii), 10% lactic acid (iii);

70% propylene glycol (i), 20% anisalcohol (i), 5% quercetin (ii), 5% tannin (ii);

82% L-menthol (i), 8% anisol (iv), 7% citronellol (i), 3% safrol (iv);

98% propylene glycol (i), 2% allicin (v).

3. Compositions comprising benzyl alcohol as a necessary component:

79% benzyl alcohol (i), 20% geraniol (i), 1% tannin (ii);

95% benzyl alcohol (i), 5% mandelic acid (iii);

70% benzyl alcohol (i), 30% catechol (ii);

1% benzyl alcohol (i), 88% propylene glycol (i), 1% tannin (ii), 10% lactic acid (iii).

4. Compositions comprising at least two GRAS essential oils (i):

90% bitter orange (ix6), 10% cinnamon (ix2);

6% origanum (ix3), 8% coriander (ix1), 7% citric acid (iii), 79% propylene glycol (carrier agent);

1% lavender (ix4), 1% anise (ix3), 2% safrol (iv), 96% xanthan (carrier agent);

5% cardamon (ix1), 5% anise (ix3), 10% eucalyptus (ix2), 80% carrier agents, especially alginic acid.

5. Compositions comprising at least one lipophilic and at least one hydrophilic GRAS flavoring agent:

25% cinnamyl alcohol (iₗ), 25% linalool (iₗ), 50% glycerol (iₕ);

25% cinnamyl alcohol (iₗ), 25% linalool (iₗ), 50% lactic acid (iiiₕ);

50% benzyl alcohol (iₗ), 50% glycerol (iₕ);

50% benzyl alcohol (iₗ), 50% lactic acid (iiiₕ);

50% tannin (ii), 50% glycerol (iₕ);

50% tannin (ii), 50% lactic acid (iiiₕ);

45% cinnamyl alcohol (i₁), 40% linalool (i₁), 10% tannin (ii), 5% vanillin (viiiₕ).

6. Compositions comprising at least one GRAS flavoring agent having a double bond:

70% cinnamic aldehyde (viii_{Δ}), 30% origanum (ix3);

20% eugenol (iv), 50% citronellol (i_{Δ}), 20% citronellal (viii_{Δ}), 10% ethylcinnamate (v_{Δ});

10% cinnamic aldehyde (viii_{Δ}), 90% glycerol (i).

Said animal and plant extracts (b3) contain at least one component with alcoholic, acid, ester, aldehyde, acetal, phenolic functional groups and/or with double bonds. Particularly preferred are phenolic, acid, alcoholic, aldehyde, double bonds. It is particularly preferred for the extracts to be of the following origins or to contain the following components:

Aloe, annatto (seeds), arnica, valerian, woundwort, birch leaves, pollen, nettle, champignon, dill, ivy, althaea, oak (bark), spruce (needles), gingko (leaves), ginseng (root), hamamelis (leaves), henna, hayflowers, hibiscus, hop, coltsfoot, ginger (root), St. John's wort, cocoa, coffee, camomile, carots, garlic, cola nut, coriander, caraway, lavender, lime (flowers), dandelion, mallows, butcher's broom, marjoram, melissa, orange (peels), orthosiphonis, paprica, pepper, peppermint, mushrooms, rhubarb, marigolds, rosmary, horse chestnuts, sage, horsetail, celandine, black tea, liquorice, juniper (berries), hawthorn, wheat bran, cinnamon, algae, banana, benzo, birch tar, trigonella, boldo, cinchona bark, copai balm, derris root, cajeput oil, gentian, eucalyptus, fennel, buckbean, sweet flag, potato starch, pine, colophonium, onion, larch turpentine, linseed, laurel, conifer tar, niduli tree, Peru balsam, pyrethrum, quassia wood, rice, rotenon, sade tree, saponin, black salsify, mustard, sesame, soybean, coneflower, centaurium, turpentine oil, plantain, white false hellebore, wormwood, cypress, apple, avocado, Theobroma grandiflorum, grapefruit, guarana, cucumber, oat, melon, jojoba, kiwi, burr, clover, coconut, cornflower, almond, mango, mistletoe, taxus, papaya, passion fruit, peach, yarrow, primrose, sunflower, dead nettle, thyme, walnut, water mint, ylang ylang, cochenille, beeswax, royal jelly, propolis, shellac, lecithin, formiates, formic acid, lutein, canthaxanthin, riboflavin, lactoflavin, edible fatty acids and derivatives, angora, astrahan, cashmere, mohair, wool, milk, casein, caseinogen, bee pollen, bee venom, honey, whey, eggs, karatin, placenta, fish, meat, fowl, ambra, capiz, caviar, chitin, coral, squalene, seal oil, whale oil, sponges, pearls, roe, seal, shellfish, sperm oil, seed oil, wax, carmine, carminic acid, catgut, musk, civet, castor, estrogen, progesterone, testerone, hormones, ivory, lanolin, mink fat, parchment, silk, snake venom, urea, amino acids, aspic, bones, bristles, collagen, down, blood, gelatin, glycerol, hide, leather, oleostearine, rennet, stearine derivatives.

In addition to components (a) and (b), the medicament may further contain an additive component (c) selected from

(c1) at least one vitamin;

(c2) at least one mineral or trace element;

(c3) at least one growth promoter or supporter;

(c4) at least one additive, especially carrier agent, separating agent or timer substance;

(c5) at least one enzyme additive.

Suitable vitamins (c1) according to the present invention are, in particular, ascorbic acid, ascorbyl palmitate, ascorbates, especially magnesium ascorbates, β-, γ- and δ-tocopherol, α- and β-carotene, thiamine, riboflavine, niacin, pantothenic acid, calcium pantothenate, pyridoxine hydrochloride, cyanocobalamine, cholecalciferol, cholesterol, biotin, folic acid and nicotinamide. Suitable minerals (c2) are, in particular, calcium, phosphorus, iron, magnesium, iodine, potassium, chlorine, copper, manganese, chromium, molybdenum, selenium, silicon and tin. Growth promoters and supporters (c3) according to the present invention include yeast, sugar (including lactose, glucose, beet and cane sugars), royal jelly, peptone, bile salts, common salt, agar, gelatin, potato extract, meat extract, citrates, bovine heart tissue, starch, nicotinic acid, tryptose, sodium azide, mannitol, magnesium sulfate, disodium hydrogenphosphate, potassium hydrogenphosphate, sodium pyruvate, casein peptone, glycine, lithium chloride, dipotassium phosphate, sodium sulfite, iron sulfate, proteose-peptone, liver digestion product, potato infusion, ox gall, sodium thioglycolate, calcium chloride, soybean flour, soybean flour peptone, L-cysteine, L-asparagine, sodium dithionite, lecithin, mineral acids and their salts, bovine and sheep serums, deoxyribonucleic acids, acetates, adenine sulfate, guanine hydrochloride, casein hydrolysate, calf brain infusion, bovine heart infusion, charcoal, liver infusion. The additives (c4) of the present invention are preferably selected from salts, sugars, carbohydrates, egg white, proteins, liquids, especially oils and water, fats, fatty acids and carrier agents, especially propylene glycol, benzyl alcohol, glycerol, alginates, lactates, silicic acid, gelatin and agar. Suitable enzyme additives (c5) are, in particular, inositol, rutin, lutein, L-arginine, L-cysteine, L-carnitine, L-lysine, L-proline and coenzyme Q10.

Preferred compositions include compositions (I) to (VI), which contain the following components:

(I) (a) at least one apathogenic microorganism (a1), preferably Lactobacillus acidophilus and/or Lactobacillus fermentum, (b) at least one enzyme (b1), preferably a hydrolase and/or protease, and optionally an additional additive component (c) selected from vitamins (c1), minerals and trace elements (c2);

(II) (a) at least one apathogenic microorganism (a1), preferably Lactobacillus acidophilus and/or Lactobacillus fermentum, (b) at least one GRAS flavoring agent or derivative thereof (b2), preferably cardamon, anise and/or tannin, and optionally an additional additive component (c) selected from vitamins (c1), minerals and trace elements (c2), and/or one or more enzymes (b1);

(III) (a) at least one apathogenic microorganism (a1), preferably Lactobacillus acidophilus and/or Lactobacillus fermentum, (b) at least one animal or plant extract or derivative thereof (b3), preferably an extract from taxus, gingko, avocado and/or bee pollen, and optionally an additional additive component (c) selected from vitamins (c1), minerals and trace elements (c2), one or more enzymes (b1), and/or one or more GRAS flavoring agents or derivatives thereof (b2);

(IV) (a) at least one pathogenic or facultatively pathogenic microorganism (a2), preferably Bacillus subtilis, Serratia marcescens and/or Streptococcus, (b) at least one enzyme (b1), preferably a hydrolase and/or protease, and optionally an additional additive component (c) selected from vitamins (c1), minerals and trace elements (c2), and/or an apathogenic microorganism (a1);

(V) (a) at least one pathogenic or facultatively pathogenic microorganism (a2), preferably Bacillus subtilis, Serratia marcescens and/or Streptococcus, (b) at least one GRAS flavoring agent or derivative thereof (b2), preferably cardamon, anise and/or tannin, and optionally an additional additive component (c) selected from vitamins (c1), minerals and trace elements (c2), and/or one or more enzymes (b1), and/or an apathogenic microorganism (a1);

(VI) (a) at least one pathogenic or facultatively pathogenic microorganism (a2), preferably Bacillus subtilis, Serratia marcescens and/or Streptococcus, (b) at least one animal or plant extract or derivative thereof (b3), preferably an extract from taxus, gingko, avocado and/or bee pollen, and optionally an additional additive component (c) selected from vitamins (c1), minerals and trace elements (c2), one or more enzymes (b1), and/or one or more GRAS flavoring agents or derivatives thereof (b2), and/or an apathogenic microorganism (a1).

The most preferred compositions are those compositions which correspond to the composition BHQ R as mentioned in the experimental part of the application (not being limited to the special content of the component).

In addition to components (a) to (c), the medicaments (1) and food supplements (6) according to the invention may contain further commercially available pharmacologically acceptable compounds and carrier materials (k), such as alcohols (k1), emulsifiers (k2), stabilizers (k3), antioxidants (k4), preservatives (k5), solvents (k6), ointments (k7), carrier agents (k8) including those having a “timer” function (i.e., disintegration=effect at the designated site) etc. The proportion of components (k) in the medicament or agent depends on the dosage form of the medicament/agent and may be up to 95% by weight, is preferably smaller than 10% by weight and is preferably within a range of from 0.1 to 5% by weight. Thus, the amount of additives is very low in inhalation agents (aerosols) with a content of microbicidal composition of as much as over 90% by weight of the aerosol, but is clearly larger, for example, when the medicament is applied orally, intravenously or intramuscularly in which case the content of microbicidal composition is usually within a range of from 0.1 to 20% by weight, but in some applications, it may also be up to above 95 and even 100% by weight of the functional composition. Similarly, the mutual proportion of compounds (k) depends on the dosage form of the medicament.

According to the invention, the alcohols (k1) are monovalent or polyvalent alcohols having from 2 to 10 carbon atoms, preferably from 2 to 7 carbon atoms, not including the GRAS alcohols (a). Preferably, GRAS flavor alcohols (a) and other alcohols (k1) are employed in such amounts that their mixing ratio is between 1000:1 and 1:1000, especially between 100:1 and 1:100, more preferably between 10:1 and 1:10.

The medicament may be in a solid, liquid or gaseous form to be administered to humans and animals. The microorganism component (a) or (a1) and/or (a2) and the modulator component (b) or (b1), (b2) and/or (b3) can be in a partially or completely separated form (heterogeneous) or mixed with each other (homogeneous). Partial separation of the components means that two or more components are mixed with each other, but separated from the other component(s). The separation of individual or several components can be effected by solid-solid and/or solid-liquid phase separations. The separation of individual or several components can have an advantageous effect on the shelf life and/or application property of the regenerative medicament and can cause a prolonged shelf life, in particular.

The regenerative medicament can be in the form of a tablet, especially as a tablet consisting of a single layer, but also one consisting of two, three, four or more layers. The respective layer can contain either one or more components selected from (a), (a1), (a2), (b), (b1), (b2) and (b3). The component(s) selected from (a), (a1) and/or (a2) preferably has/have a water content within a range of from 0.01 to 99% by weight. However, water contents of from 0.1 to 20% by weight are preferred. When applied as a liquid, concentrates having the above mentioned water content are preferred.

The medicament may be an inhalational, oral, intravenous, intramuscular, rectal agent, contact preparation, internal/external (also mucosa), intraperitoneal, subcutaneous agent, displaying its activity on/in internal organs (e.g., by endoscopy) in the form of tablets, liquid, gas, powder, injection, infusion, suppository, spray, ointments, plasters. The medicament may be for preventive administration and for the treatment of acute affliction.

According to embodiment (4), the invention further relates to the use of the above defined microbicidal composition (1) or (2) for preparing agents for treating humans and animals, especially for bronchitis, pneumonia (inhalative).

Further, the invention relates to methods for treating immune diseases and infectious diseases in humans and animals (5), for example, the treatment of gastritis, colitis ulcerosa, etc.

The required dose is dependent on the kind and severity of the disease, age, sex, weight and general health condition of the patient, and is usually within a range of from 0.1 to 1000 mg, preferably from 1 to 10 mg, per kg of body weight of the patient per day.

Such treatment may be effected according to the following treatment schemes I to III:

I. Multi-step:

1. “Decontaminative agent” for destroying pathogens or infection-causing agents in or on the body with flavor-containing medicaments (depending on the formulation and dosage).

2. (a) “Regenerative agent” for regenerating (maintaining or growth promotion) of necessary microorganisms in or on the body with flavor-containing medicaments (depending on the formulation and dosage).

(b) Optionally adding simultaneously or thereafter “positively necessary” microorganisms (e.g., symbiotic regenerative agent with microorganisms (singly or in admixture), and/or extracts from plants and animals and/or vitamins (singly or in admixture), and/or minerals (singly or in admixture), and/or enzymes (singly or in admixture), but depending on the formulation and dosage).

II. Combinatory method:

1. +2. (a)+(b) simultaneously

1. +2. (a)

1. +2. (b)

2. (a)+2. (b)

III. Individual application: 1, 2a, 2b and/or combinatory or multi-step with medicaments of the prior art (e.g., anti-infective agents, antidepressants, cytostatic agents, contraceptives, and many more).

The food supplements and animal feeds/feed supplements (6) of the present invention preferably contain from 0.1 to 20% by weight of the composition of components (a), (b) and (c), but in some applications, the content may also be up to 95 or even 100% by weight of the functional composition.

In the method for stabilizing cultures of microorganisms, the GRAS flavoring agent is added to the culture to be stabilized in the amounts defined above, preferably in amounts of from 10 μg to 80 mg per ml of culture solution or per mg of culture lyophilisate.

The present invention is further illustrated by means of the following Examples.

### EXAMPLES

Materials and Methods

The stated amounts in the following formulations are in percent by weight unless otherwise specified.

BHQ R11: Microorganisms (Apathogenic/Facultatively Pathogenic/Pathogenic), Enzymes, Vitamins, Minerals

Indication: gastro-intestinal diseases (diarrhea, colitis and the like)

Application: orally (1.5 g/tablet)

Lactobacillus bifidus 10¹¹, Lactobacillus acidophilus 10¹², Bacillus subtilis 10⁸, E. coli 10⁴, enzymes: L-proline 3.5%, L-lysine 3.5%, L-carnitine 1.2%, L-arginine 1.5%, trypsin 3.0%, bromelain 1.8%, vitamins: C 20%, E 2.5%, A 15%, B1, B2, B3, B5, B6, B12, minerals: calcium, chromium, copper, manganese, magnesium, selenium, zinc, phosphorus, carrier agents ad 100%.

BHQ R8: Microorganisms (Apathogenic), Flavor Synergists, Essential Oils

Indication: diseases of the respiratory tract (bronchitis, pneumonia and the like)

Application: inhalatory

Lactobacillus bifidus 10¹³, Lactobacillus acidophilus 10¹², flavors (essential oils): 1% origanum, 0.5% thyme, 0.1% camphor, 0.5% anise, 1% estragon, 2% nutmeg, 0.1% melissa, 1% cardamon, 1% fennel, 5% benzyl alcohol, propylene glycol ad 100%.

BHQ R15: Microorganisms (Apathogenic/Facultatively Pathogenic/Pathogenic), Flavor Synergists, Enzymes, Plant Extracts, Vitamins, Minerals

Indication: possible tumoricides/cytostatic effect in tumor and cancer diseases

Application: orally

Lactobacillus acidophilus 10¹⁴, Bacillus subtilis 10⁸, Enterococcus d. 10⁶, essential oils: 5% cardamon, 5% anise, 10% Eucalyptus citriodora, enzymes: 1% amylase, 0.01% leucoprotease, 0.01% trypsin, vitamins, minerals, plant extract, 0.1% cinchona bark (60 T), gingko (40 T), propylene glycol in carrier agent ad 100% (tablet, 20% active ingredient).

BHQ R7: Microorganisms (Apathogenic), Plant Extract, Vitamins, Minerals

Indication: allergic skin diseases (neurodermitis, eczemas and the like)

Application: ointment, externally

Lactobacillus acidophilus 10¹⁴, Bacillus subtilis 10¹⁰, plant extract: aloe 3%, algae 5%, larch turpentine 0.1%, sesame 1%, lecithin 0.5%, avocado 2%, vitamins, minerals, ointment base ad 100%.

BHQ R13: Microorganisms (Apathogenic), Flavor Synergists (GRAS Alcohols, Polyphenol), Vitamins, Minerals

Indication: allergic diseases (neurodermitis, asthma and the like)

Application: orally (liquid)

Lactobacillus acidophilus 10¹⁶, flavors: 1% benzyl alcohol, 3% tannin, 10% lactic acid, 3% vitamins, 3% minerals, 80% propylene glycol.

BHQ R6: Microorganisms (Apathogenic/Facultatively Pathogenic), Flavor Synergists, Enzymes

Indication: depression

Application: orally

Lactobacillus brevis 10¹², Lactobacillus acidophilus 10¹⁶, Lactobacillus bifidus 10¹², Lactobacillus bifidolongum 10¹⁴, Serratia marcescens 10⁵, flavor synergists: 20% benzyl alcohol, 5% cardamon, 3% tannin, 2% Eucalyptus citriodora, enzymes: 5% trypsin, 5% papain, 1% chymosin, 2% ficain, 2% ananain, 55% carrier agent (30% active ingredient in carrier agent, tablet 1.5 g).

BHO R16: Microorganisms (Apathogenic), Enzymes, Vitamins

Indication: digestive insufficiency, food intolerance, lack of appetite, non-specific gastro-intestinal complaints

Application: orally

Lactobacillus brevis 10⁶, Lactobacillus acidophilus 10¹⁵, Lactobacillus bifidus 10¹⁸, Lactobacillus bifidolongum 10¹⁴, enzymes: 12% total, equal portions each of: 15% amylase, protease mixture 85% (18 different enzymes), 3% bromelain, 5% papain, vitamins 3%, carrier agent ad 100% (tablet 2.5 g).

BN 3

### Comparative Example

3 kinds of lactobacilli from the groups acidophilus, bifidus, bifidolongum

13 vitamins (A, E, C, K, B1, B2, B6, B12, D, biotin, folic acid, nicotinamide, pantothenic acid)

14 minerals/trace elements (Ca, P, Fe, Mg, I, K, Cl, Cu, Mn, Cr, Mo, Se, Si, Zn)

### Example 1

The following demonstrations of action and tests were performed for the reasons described as follows:

The increase of the growth rates of broiler chicks is a test criterion for regenerative medicaments. In breeding and factory farming, broiler chicks tend to clinical pictures such as diarrhea or bronchitis, which ultimately leads to growth and/or weight losses in the breeding phase, so that the weight difference between the untreated and treated broiler chicks is a test criterion. It is known that most broiler chicks today must be treated by feed additives and/or vaccination antibiotically and/or vaccine-specifically against diseases and the accompanying weight loss.

TABLE 1
Effectiveness against gastro-intestinal diseases

| | Slaughtering result, g/broiler | | |
|---|---|---|---|
| | chick Treatment day | | |
| | 1 to day 28 Feed supplement | | Surplus weight |
| Stable, number of | | 40 mg BHQ | due to BHQ |
| broiler chicks | untreated | R11/kg | treatment |
| 12,000 stable | average | average | 152 g/animal |
| untreated | slaughtering | slaughtering | |
| | weight 1350 g/ | weight 1502 g/ | |
| | animal | animal | |
| 13,100 stable | | | |
| treated with BHQ, | | | |
| predominant | | | |
| clinical picture: | | | |
| diarrhea | | | |

The broiler chicks present in a stable were fed daily with, on the one hand, fodder (untreated) and, on the other hand, fodder and supplement (40 mg BHQ R11/kg of fodder) from day 1 to day 28 of the breeding. Result: The untreated broiler chicks suffered from an essentially higher diarrhea rate as compared to the broiler chicks treated with BHQ R11, for which a higher mast (slaughtering) weight could be clearly established. Despite of regular fodder uptake by the broiler chicks, the diarrhea specification can be seen from the weight loss.

TABLE 2
Effectiveness against diseases of the respiratory tract and/or lung
(bronchial diseases/pneumonias); inhalatory administration

| | Slaughtering result, g/broiler chick | | |
|---|---|---|---|
| | Treatment day 1 to day 28 | | Surplus weight |
| Stable, number of | | daily 10 ml | due to BHQ |
| broiler chicks | untreated | of BHQ R8 | treatment |
| 28,000 stable | 1389 g/ | nebulized into the | 94 g/animal |
| untreated | animal | stable with Atomist in | |
| | | 7 l water per 10,000 | |
| | | broiler chicks (with | |
| | | closed aeration) | |
| 25,000 stable | | 1483 g/animal | |
| treated with BHQ, | | | |
| predominant | | | |
| clinical picture: | | | |
| bronchitis, | | | |
| pneumonia, | | | |
| lethal cases | | | |

The broiler chicks present in such a stable were treated, on the one hand, with BHQ by nebulizing with an Atomist device in the stable air (once daily, nebulization with closed aeration, 10 ml/10,000 chicks, day 1 to day 28), the other case remained untreated. Result: The untreated broiler chicks suffered from audible respiration noises, LS pneumonia, pertonitis and the like, which manifested itself in a lesser fodder uptake, so that an underweight could be established on average. The BHQ-treated broiler chicks were healthier and heavier.

### Example 2

Effectiveness in cancer diseases (1 proband test)

Oral administration of BHQ in secondary carcinoma (bone tumor), osteoplastic metastases from mamma carcinoma

After a mamma carcinoma surgery, osteoplastic metastases developed in a patient (43 years); being a secondary bone (tumor) carcinoma, it was treated in a conservative way by chemotherapy and irradiation (level 1). After the cytostatic agents were discontinued, the irradiations were continued due to a risk of leucopenia, with oral administration (combinatory) of BHQ R15. After 3 months, a subjective improvement as well as an improvement of the clinical data was observed (level 2).

TABLE 3

| Symptoms | Level 0 | Level 1 | Level 2 |
|---|---|---|---|
| General health condition | − | − | +/− |
| Appetite | − | − | +/− |
| Pain | − | − | − |
| Fever | + | + | − |
| Alkaline phosphatase | 800 μg/ | 280 μg/100 ml | 200 μg/100 ml |
| (ALP)* | 100 ml | | |
| Leucocytosis** | 22,000 mm³ | 14,000 mm³ | 10,500 mm³ |
| (leucocyte proliferation) | | | |

*ALP 40 190 normal
**Leucocytes > 10,000 mm³ = leucocytosis (<5,000 = leucopenia (leucocyte reduction))
(−) negative
(+) positive
Level 0 - disease has started
Level 1 - treatment: chemotherapy/irradiation
Level 2 - treatment: irradiation with oral administration of 3-5 g/day (3 months) of BHQ R15

Conclusion: Combinatory accompanying BHQ administration resulted in a clear improvement and well-being of the patient.

### Example 3

Effectiveness in allergies (3 proband tests)

Oral and contact skin preparation (external) BHQ administrations in neurodermitis atopica (chronic recurrent skin eczema, “eczema flexuarum”, in the bends of joints in adults (32-40-46 years).

A substantial improvement as compared to the “conservative” therapy could be achieved with BHQ applied orally and externally.

Three probands suffered from recurrent skin eczemas in the bends of their joints. A conservative treatment with antihistaminic agents and corticoid-containing ointments did not result in a permanent relief of the itching or permanent elimination of the eczematous skin regions (level 0). The conservative therapy was replaced by an oral administration of BHQ R13 for 30 days. Two probands did not have any more visible eczemas and did not feel any more itching (level 1). Then, the probands were also treated with BHQ R7 ointment externally at the skin regions twice a day over 20 days. All symptoms had vanished (level 2).

TABLE 4

| Symptoms | | | |
|---|---|---|---|
| Eczema | Level 0 | Level 1 | Level 2 |
| 32 years (f) | + | +/− | − |
| 40 years (m) | + | − | − |
| 46 years (m) | + | − | − |

+ eczemas present
+/− improvement
− eczemas no longer detectable, accompanying phenomena vanished

Level 0: disease has started, treatment symptomatic with antihistaminic agents, glucocorticoid and glucocorticoid-containing external agents

Level 1: discontinuation of conservative treatment, 30 days oral administration with BHQ R13 (once daily, 1.3 g)

Level 2: treatment after 20 days with oral administration of BHQ (level 1) with additional external agents (ointment preparation), BHQ R7 treatment of the eczematous skin regions over two weeks (twice daily). Six months later, no eczemas had occurred any more.

### Example 4

Antidepressive effectiveness (2 proband tests/female 1/male 1)

Diseases such as depressions can often be treated in an exclusively symptomatic way with psychopharmaceuticals. However, mycoses/mycotoxins may be the initial cause of such diseases. The object was to eliminate the gastro-intestinal over-growth with Aspergillus and Candida yeasts using BHQ (see also Patent Application EP 00 124 497), and to apply BHQ R6 regenerative agent in a multi-step method. After application of the BHQ therapy in a multi-step method, all three probands were free of depression.

TABLE 5

| Symptoms | Level 0 | Level 1 | Level 2 |
|---|---|---|---|
| General health | − | +/− | + |
| Weight increase | + | +/− | − |
| Feelings of anxiety/ | +/− | +/− | − |
| depression | | | |
| part. status | + | − | − |
| microbiological | + | − | − |
| gastro-intestinal | | | |
| detection: | | | |
| Candida spec. | | | |
| Aspergillus spec. | | | |

− negative
+ positive

Level 0: Depression conservatively treated with antidepressants (Seroxat®). The general state of health was subjectively reported as non-satisfactory; in part, depression attacks occurred at different times of day (e.g., in the evening), so that the dosage was increased. The microbiological detection of Candida spec. and Aspergillus spec. was positive. The probands complained about a high weight increase (“spongy feeling”).

Level 1: For 4 weeks, BHQ “flavor-containing medicament” was administered orally once a day for the decontamination of the mycosis and/or any mycotoxins present (1 g/day). As seen from the Table, an improvement could be observed already after 4 weeks.

Level 2: As a next step after level 1, BHQ R6 regenerative agent (2 g daily) was administered orally for 4 weeks. After the treatment, the subjects were free of complaints and reduced their weights.

### Example 5

Effectiveness in clinical pictures produced from gallbladder resection and peritonitis with deficient production of bile acid and enzymes. A proband (female) permanently suffered from lack of appetite, intolerance towards non-dietary food, subjective non-specific gastro-intestinal complaints, and lack of physical and psychical stress tolerance.

TABLE 6

| Symptoms | Level 0 | Level 1 |
|---|---|---|
| General health | − | + |
| Appetite | − | + |
| Food tolerance | − | +/− |
| non-spec. gastro-intestinal | − | + |
| complaints | | |
| physical stress tolerance | − | + |
| psychical stress tolerance | − | + |

− negative
+ positive

Level 0: The proband suffered from the symptoms stated in Table 6 after a gallbladder resection to which she had been subjected 1 year ago and peritonitis. Various conservative medicament administrations did not result in a subjectively felt general positive state of health.

Level 1: BHQ R16 also contains enzymes as a regenerative agent. The therapy was effected orally with daily administrations (1.5 g) over a period of 90 days. After this period, the proband was free of complaints.

### Example 6

### Food

Effectiveness: see Table 7, suspension experiments “BN 3” with comparative composition

Under gastro-intestinal conditions (37° C.), the germ count increases after a time of action of 6 hours from 24,000 cfu/ml (colony-forming units/ml) with Lactobacillus acidophilus to 26,000 cfu/ml with “BN 3” at a dosage of 0.10%. However, after 24 hours of action, this weak colonization is ruined by the fact that the growth control (without “BN 3”) increases to 43,000 cfu/ml, but the Lactobacillus acidophilus treated with “BN 3” are decreased by 90% (log RF 1.0) to 4,300, i.e., after a short time of stimulation, colonization, “BN 3” only has short-term regenerative properties, then ruining its effect in an extended time of action (digestion process about 16 to 24 h) and exerting an even very highly decontaminative rather than regenerative effect on the “probiotic microorganisms” to be introduced, i.e., does not reach its regenerative properties over one day. The idea of these preparations is to achieve “docking” of “probiotic microorganisms” (e.g., of the Lactobacillus group) to the mucosa formulations of the gastro-intestinal tract by the addition of vitamins and/or minerals through stimulation or colonization of the microorganisms. According to the evaluation of the chosen test criterions, this is not successful here with oral administration of a dose once a day.

TABLE 7
Quantitative suspension experiment

Test germ: Lactobacillus acidophilus DSM 20079
cfu/ml: 3.7 · 10⁷
Test conditions: test temperature 37° C.
Medium: MRS agar, MRS broth, pH 8.0 + Comp. IV (3% Tween ® +
0.3% lecithin + 0.1% histidine + 3% saponin)

| | Conc. in % | Time of | Control | Time of | Control |
|---|---|---|---|---|---|
| Product | by volume | action 6 h | 37° C. | action 24 h | 37° C. |
| BHQ R15 | 0.1 | 84,000 | 24,000 | 55,000 | 43,000 |
| BHQ R6 | 0.1 | 70,000 | 24,000 | 43,000 | 43,000 |
| BHQ R13 | 0.1 | 61,000 | 24,000 | 40,000 | 43,000 |
| BHQ R8 | 0.1 | 56,000 | 24,000 | 38,000 | 43,000 |
| BHQ R11 | 0.1 | 52,000 | 24,000 | 31,000 | 43,000 |
| BHQ R16 | 0.1 | 48,000 | 24,000 | 34,000 | 43,000 |
| BHQ R7 | 0.1 | 37,000 | 24,000 | 24,000 | 43,000 |
| BN 3* | 0.1 | 26,000 | 24,000 | 4,300 | 43,000 |

*Comparative Example

A composition of apathogenic microorganisms and vitamins/minerals according to the prior art “BN 3” has a slight colonizing effectiveness after 6 h (37° C.) of action, which exhibits a decontaminative effect, i.e., the reverse effect, within 24 h. Under the synergistic effect of flavor, enzyme and plant extract and vitamins/minerals, the symbiotic complex of apathogenic, facultatively pathogenic and pathogenic microorganisms colonizes over 6 and 24 hours (37° C.). Once flavor synergisms with microorganisms have a symbiotic effect, they exhibit a colonization which is above the 6 h or 24 h control value even after 24 h. Enzymes show a similar, but weaker effect.

1. A regenerative medicament, comprising:
(a) a microorganism component containing:
(a1) at least one type of apathogenic microorganisms; and/or
(a2) at least one type of pathogenic or facultatively pathogenic microorganisms;
(b) a modulator component containing
(b1) at least one enzyme;
(b2) at least one GRAS (generally recognized as safe) flavoring agent or a derivative thereof; and/or
(b3) at least one animal or plant extract or derivative thereof which comprises a compound with alcoholic, acid, ester, aldehyde, acetal, phenolic functional groups and/or with double bonds.

2. The medicament according to claim 1, wherein said apathogenic microorganism (a1) is an apathogenic lactobacillus, preferably selected from Lactobacillus acidophilus, Lactobacillus bifidum, Lactobacillus bulgaris, Lactobacillus casei, Lactobacillus bifidolongum and Lactobacillus fermentum, and/or a microorganism selected from fungi, dermatophytes and yeasts which do not have pathogenic or toxinogenic properties or which occur in the gastrointestinal tract, including Sarcina, Staphylococcus epidermis, Gaffkya, Streptococcus K-P, Corynebacterium xerosis, Corynebacterium hoffmanni, Bacillus subtilis and Saccharomyces cerevisiae; and/or
said pathogenic or facultatively pathogenic microorganisms (a2) are selected from Serratia marcescens, Alcaligenes faecalis, Citrobacter, Hafnia alvei, Afflya tetragena, Neisseria catarrhalis, Neisseria sicca, Neisseria flavescens, subflava, flava, perflava, Veillonella parvula, Borrelia buccalis, Rhodotorula rubra, Cladosporium, Geotrichum mucor, Mucor and Torulopsis;
wherein said microorganism component preferably contains at least two, more preferably at least three different microorganisms.

3. A regenerative medicament according to one or more of claims 1 to 3, wherein the enzyme (b1) is of vegetable, animal or synthetic origin, in particular, selected from chymotrypsin, bromelain, papain, pepsin, trypsin, an enzyme from the groups of oxidoreductases, dehydrogenases, oxygenases, transferases, hydrolases, esterases, glycosidases, proteases, lyases, isomerases, ligases, synthetases, DNA ligases, amylases, lipases, phosphatases, leucoproteases, cathepsins, oxidases, such as catalases and peroxidases, carboxylases, more preferably a hydrolase or proteases, such as bromelain, trypsin, ananain, pepsin, chymosin, ficain; and/or
said (generally recognized as safe) flavoring agent or its derivative (b2) is selected from:
(i) GRAS flavor alcohols or their derivatives, especially benzyl alcohol, acetoin, propyl alcohol, iso-propyl alcohol, propylene glycol, glycerol, n-butyl alcohol, iso-butyl alcohol, hexyl alcohol, L-menthol, octyl alcohol, cinnamyl alcohol, α-methylbenzyl alcohol, heptyl alcohol, n-amyl alcohol, iso-amyl alcohol, anisalcohol, citronellol, n-decyl alcohol, geraniol, β-γ-hexanol, lauryl alcohol, linalool, nerolidol, nonadienol, nonyl alcohol, rhodinol, terpineol, borneol, clineol, anisole, cuminyl alcohol, 10-undecene-1-ol, 1-hexadecanol and their derivatives;
(ii) polyphenol compounds, especially catechol, resorcinol, hydroquinone, phloroglucinol, pyrogallol, cyclohexane, usnic acid, acylpolyphenols, lignins, anthocyans, flavones, catechols, gallic acid derivatives, caffeic acid, flavonoids, derivatives of the mentioned polyphenols, and extracts from Camellia, Primula;
(iii) GRAS acids or derivatives, especially acetic acid, aconitic acid, adipic acid, formic acid, malic acid, capronic acid, hydrocinnamic acid, pelargonic acid, lactic acid, phenoxyacetic acid, phenylacetic acid, valeric acid, iso-valeric acid, cinnamic acid, citric acid, mandelic acid, tartaric acid, fumaric acid, tannic acid and their derivatives;
(iv) GRAS phenols or their derivatives, especially thymol, methyleugenol, acetyleugenol, safrol, eugenol, isoeugenol, anethole, phenol, methylchavicol, carvacrol, α-bisabolol, formesol, anisole, propenylguaethol and their derivatives;
(v) GRAS esters, especially acetates including iso-amyl acetate, benzyl acetate, benzylphenyl acetate, n-butyl acetate, cinnamyl acetate, citronellyl acetate, ethyl acetate, eugenol acetate, geranyl acetate, hexyl acetate, hydrocinnamyl acetate, linalyl acetate, octyl acetate, phenylethyl acetate, terpinyl acetate, triacetin, potassium acetate, sodium acetate and calcium acetate as well as esters and ester derivatives of acids (iii);
(vi) terpenes, especially camphor, limonene and β-caryophyllene;
(vii) acetals, especially acetal, acetaldehyde dibutyl acetal, acetaldehyde dipropyl acetal, acetaldehyde phenethyl propyl acetal, cinnamic aldehyde ethylene glycol acetal, decanal dimethyl acetal, heptanal dimethyl acetal, heptanal glyceryl acetal and benzaldehyde propylene glycol acetal;
(viii) aldehydes, especially acetaldehyde, anisaldehyde, benzaldehyde, iso-butyl aldehyde, citral, citronellal, n-caprylic aldehyde, ethylvanillin, furfural, heliotropin, heptyl aldehyde, hexyl aldehyde, 2-hexenal, hydrocinnamic aldehyde, lauryl aldehyde, nonyl aldehyde, octyl aldehyde, phenylacetaldehyde, propionaldehyde, vanillin, cinnamic aldehyde, perillaldehyde and cuminaldehyde;
(ix) GRAS essential oils, especially essential oils and/or alcoholic or glycolic extracts or extracts obtained by CO₂ high-pressure processes from the following plants:
(ix1) oils or extracts having a high content of alcohols: melissa, coriander, cardamon, eucalyptus;
(ix2) oils or extracts having a high content of aldehydes: Eucalyptus citriodora, cinnamon, lemon, lemon grass, melissa, citronella, lime, orange;
(ix3) oils or extracts having a high content of phenols: origanum, thyme, rosemary, orange, clove, fennel, camphor, mandarin, anise, cascarilla, estragon and pimento;
(ix4) oils or extracts having a high content of acetates: lavender;
(ix5) oils or extracts having a high content of esters: mustard, onion, garlic;
(ix6) oils or extracts having a high content of terpenes: pepper, bitter orange, caraway, dill, lemon, peppermint, nutmeg; and/or
said animal or plant extract (b3) is selected from extracts of aloe, annatto (seeds), arnica, valerian, woundwort, birch leaves, pollen, nettle, champignon, dill, ivy, althaea, oak (bark), spruce (needles), gingko (leaves), ginseng (root), hamamelis (leaves), henna, hayflowers, hibiscus, hop, coltsfoot, ginger (root), St. John's wort, cocoa, coffee, camomile, carots, garlic, cola nut, coriander, caraway, lavender, lime (flowers), dandelion, mallows, butcher's broom, marjoram, melissa, orange (peels), orthosiphonis, paprica, pepper, peppermint, mushrooms, rhubarb, marigolds, rosmary, horse chestnuts, sage, horsetail, celandine, black tea, liquorice, juniper (berries), hawthorn, wheat bran, cinnamon, algae, banana, benzo, birch tar, trigonella, boldo, cinchona bark, copai balm, derris root, cajeput oil, gentian, eucalyptus, fennel, buckbean, sweet flag, potato starch, pine, colophonium, onion, larch turpentine, linseed, laurel, conifer tar, niduli tree, Peru balsam, pyrethrum, quassia wood, rice, rotenon, sade tree, saponin, black salsify, mustard, sesame, soybean, coneflower, centaurium, turpentine oil, plantain, white false hellebore, wormwood, cypress, apple, avocado, Theobroma grandiflorum, grapefruit, guarana, cucumber, oat, melon, jojoba, kiwi, burr, clover, coconut, cornflower, almond, mango, mistletoe, taxus, papaya, passion fruit, peach, yarrow, primrose, sunflower, dead nettle, thyme, walnut, water mint, ylang ylang, cochenille, beeswax, royal jelly, propolis, shellac, lecithin, formiates, for mic acid, lutein, canthaxanthin, riboflavin, lactoflavin, edible fatty acids and derivatives, angora, astrahan, cashmere, mohair, wool, milk, casein, caseinogen, bee pollen, bee venom, honey, whey, eggs, karatin, placenta, fish, meat, fowl, ambra, capiz, caviar, chitin, coral, squalene, seal oil, whale oil, sponges, pearls, roe, seal, shellfish, sperm oil, seed oil, wax, carmine, carminic acid, catgut, musk, civet, castor, estrogen, progesterone, testerone, hormones, ivory, lanolin, mink fat, parchment, silk, snake venom, urea, amino acids, aspic, bones, bristles, collagen, down, blood, gelatin, glycerol, hide, leather, oleostearine, rennet, stearine derivatives.

4. The medicament according to one or more of claims 1 to 3, wherein said medicament further contains an additive component (c) which is selected from
(c1) at least one vitamin;
(c2) at least one mineral or trace element;
(c3) at least one growth promoter or supporter;
(c4) at least one additive, especially carrier agent, separating agent or timer substance;
(c5) at least one enzyme additive;
wherein preferably
said vitamins (c1) are selected from ascorbic acid, ascorbyl palmitate, ascorbates, especially magnesium ascorbates, β-, γ- and δ-tocopherol, α- and β-carotene, thiamine, riboflavine, niacin, pantothenic acid, calcium pantothenate, pyridoxine hydrochloride, cyanocobalamine, cholecalciferol, cholesterol, biotin, folic acid and nicotinamide; and/or
said minerals and trace elements (c2) are selected from calcium, phosphorus, iron, magnesium, iodine, potassium, chlorine, copper, manganese, chromium, molybdenum, selenium, silicon and tin; and/or
said growth promoters and supporters (c3) are selected from yeast, sugar (including lactose, glucose, beet and cane sugars), royal jelly, peptone, bile salts, common salt, agar, gelatin, potato extract, meat extract, citrates, bovine heart tissue, starch, nicotinic acid, tryptose, sodium azide, mannitol, magnesium sulfate, disodium hydrogenphosphate, potassium hydrogenphosphate, sodium pyruvate, casein peptone, glycine, lithium chloride, dipotassium phosphate, sodium sulfite, iron sulfate, proteose-peptone, liver digestion product, potato infusion, ox gall, sodium thioglycolate, calcium chloride, soybean flour, soybean flour peptone, L-cysteine, L-asparagine, sodium dithionite, lecithin, mineral acids and their salts, bovine and sheep serums, deoxyribonucleic acids, acetates, adenine sulfate, guanine hydrochloride, casein hydrolysate, calf brain infusion, bovine heart infusion, charcoal and liver infusion; and/or
said additives (c4) are selected from salts, sugars, carbohydrates, egg white, proteins, liquids, especially oils and water, fats, fatty acids and carrier agents, especially propylene glycol, benzyl alcohol, glycerol, alginates, lactates, silicic acid, gelatin and agar; and/or
said enzyme additives (c5) are selected from inositol, rutin, lutein, L-arginine, L-cysteine, L-carnitine, L-lysine, L-proline and coenzyme Q10.

5. The medicament according to one or more of claims 1 to 4, wherein said microorganism component (a) contains at least one apathogenic microorganism (a1), preferably Lactobacillus acidophilus and/or Lactobacillus fermentum, said modulator component (b) contains at least one enzyme (b1), preferably a hydrolase and/or protease, and the medicament optionally contains an additional additive component (c) selected from vitamins (c1), minerals and trace elements (c2).

6. The medicament according to one or more of claims 1 to 4, wherein
(i) said microorganism component exclusively consists of apathogenic microorganisms (a1); and/or
(ii) said modulator component contains at least one GRAS flavoring agent or derivative thereof.

7. The medicament according to one or more of claims 1 to 4, wherein said microorganism component (a) contains at least one apathogenic microorganism (a1), preferably Lactobacillus acidophilus and/or Lactobacillus fermentum, said modulator component (b) contains at least one GRAS flavoring agent or derivative thereof (b2), preferably cardamon, anise and/or tannin, and the medicament optionally contains an additional additive component (c) selected from vitamins (c1), minerals and trace elements (c2), and/or one or more enzymes (b1).

8. The medicament according to one or more of claims 1 to 4, wherein said microorganism component (a) contains at least one apathogenic microorganism (a1), preferably Lactobacillus acidophilus and/or Lactobacillus fermentum, said modulator component (b) contains at least one animal or plant extract or derivative thereof (b3), preferably an extract from taxus, gingko, avocado and/or bee pollen, and the medicament optionally contains an additional additive component (c) selected from vitamins (c1), minerals and trace elements (c2), one or more enzymes (b1), and/or one or more GRAS flavoring agents or derivatives thereof (b2).

9. The medicament according to one or more of claims 1 to 4, wherein said microorganism component (a) contains at least one pathogenic or facultatively pathogenic microorganism (a2), preferably Bacillus subtilis, Serratia marcescens and/or Streptococcus, one or more modulator components (b) and optionally additional additive components (c) as defined in claims 5 to 7 and optionally contains an apathogenic microorganism (a1).

10. The medicament according to one or more of claims 1 to 9, wherein the number of microorganisms (a) in the medicament is from 10¹ to 10²⁰, preferably from 10⁶ to 10¹⁴, per g or per ml of medicament, and/or the content of (b) and (c) in total is from 1 μg to 200 mg, preferably 10 μg to 80 mg each per g or per ml of medicament.

11. The medicament according to one or more of claims 1 to 10, which further contains monovalent or polyvalent alcohols having from 2 to 10 carbon atoms, emulsifiers, stabilizers, antioxidants, preservatives, solvents and/or carrier agents.

12. The medicament according to one or more of claims 1 to 11, wherein the medicament can be applied by inhalation, orally, intravenously, intramuscularly, rectally, as a contact preparation, internally/externally, intraperitoneally, subcutaneously, on/in internal organs, in the form of tablets, liquid, gas, powder, injection, infusion, suppository, spray, ointments or plasters, and/or wherein the medicament is an immune regulator, immunoregenerative agent, anti-infective agent, antimycotic agent, antitoxinocidic agent, an tiresistance agent or cytostatic agent.

13. Use of a composition consisting of a microorganisms component (a) and a modulator component (b) as defined in claims 1 to 9 for the preparation of a decontaminative and/or regenerative agent, especially an immune regulator, immunoregenerative agent, anti-infective agent, antimycotic agent, an titoxinocidic agent, antiresistance agent or cytostatic agent, for the treatment of humans and animals.

14. A method for the treatment of non-specific genesis, allergies, cancer diseases, symptoms which are not causally associated with clinical pictures susceptible to regenerative therapy, of immune diseases and infectious diseases in humans and animals, comprising the administration of a composition consisting of a microorganism component (a) and a modulator component (b) as defined in one or more of claims 1 to 9.

15. Food, food supplements, animal feeds or animal feed supplements comprising a composition consisting of a microorganism component (a) and a modulator component (b) as defined in one or more of claims 1 to 9.

16. The food, food supplements, animal feeds or animal feed supplements according to claim 15, containing from 0.1 to 900 mg, preferably from 1 to 200 mg, of said composition of (a), (b) and (c) per g of food, food supplement, animal feed or animal feed supplement.

17. A method for the stabilization of cultures of microorganisms, comprising the treatment, storage and/or culturing of said culture of microorganisms with or in the presence of at least one GRAS flavoring agent or derivative thereof as defined in claim 1 or 3.

18. The method according to claim 17, wherein said culture of microorganisms is a microorganism component as defined in claims 1, 2 and 5 to 9.
